(19) United States
(12) Patent Application Publication
(10) Pub. No.: US 2026/0260642 A1
(43) Pub. Date: Sep. 3, 2026

(54) METHOD FOR DESIGNING CAUSALLY OPTIMAL BROADBAND ACOUSTIC METAMATERIAL ABSORBER, AND ABSORBER

(71) Applicant: ACOUSTIC METAMATERIALS COMPANY LIMITED, Hong Kong (CN)

(72) Inventors: Min YANG, Hong Kong (CN); Shuyu CHEN, Hong Kong (CN); Songwen XIAO, Hong Kong (CN); Yunfei XU, Hong Kong (CN)

(21) Appl. No.: 19/154,513

(22) PCT Filed: Apr. 11, 2024
(86) PCT No.: PCT/CN2024/087350

§ 371 Date: Aug. 7, 2025

Related U.S. Application Data
(60) Provisional application No. 63/444,966, filed on Feb. 12, 2023.

(51) Int. Cl. G10K 11/172; G06F 30/20; G06F 119/10; G10K 11/162

(52) U.S. Cl. CPC G10K 11/172; G06F 30/20; G10K 11/162; G06F 2119/10

(57) ABSTRACT

A method for designing and manufacturing a causally optimal broadband acoustic metamaterial absorber and an absorber. The method involves: (1) calculating a COBA spectrum by solving an optimization problem based on a causal constraint; (2) designing an array of resonators, the modal density and resonance strength of which match the COBA spectrum; and (3) using a mass production method, such as a molding process, to manufacture the absorber. As a result, compared with traditional porous absorbers, a produced acoustic metamaterial absorber shows a better broadband absorption performance under a given thickness constraint, particularly within a low frequency range. The present invention represents a significant advancement in the development of a high-performance and space-saving noise control solution.

### CROSS-REFERENCE TO RELATED APPLICATIONS

The present application is a National Stage of International Application No. PCT/CN2024/087350, filed on Apr. 11, 2024, and claims the benefit of priority to U.S. Provisional Application No. 63/444,966, filed on Feb. 12, 2023, both of which are incorporated herein by reference in their entireties for all purposes.

### TECHNICAL FIELD

The present disclosure relates to the technical field of sound absorbing materials, and particularly relates to a method for designing a causally optimal broadband acoustic metamaterial absorber and an absorber.

### BACKGROUND OF THE INVENTION

Noise pollution is a persistent issue in modern society, affecting human health, productivity, and quality of life. Traditional porous sound-absorbing materials such as foam and fibers have been widely used for noise control but face limitations in low-frequency absorption and performance in confined spaces. These materials absorb sound energy based on high dissipation, leading to poor performance at a low frequency, and need a thicker structure to achieve sufficient absorption.

In recent years, acoustic metamaterials have emerged as promising alternatives to the traditional sound-absorbing materials. The artificially designed structures possess a unique attribute, such as an extraordinary absorption capability not found in natural materials, due to their sub-wavelength property. The acoustic metamaterials may increase energy density by using local resonances, and improve absorption performance even at low frequencies. However, a typical design of an acoustic metamaterial absorber exhibits a narrow frequency band absorption nature, limiting application effectiveness of the acoustic metamaterial absorber in a real world that generally involves broad frequency band noise.

To solve this issue, researchers have explored various strategies to expand an absorption bandwidth of the acoustic metamaterial. One method is to integrate a plurality of resonators with different resonant frequencies into a single structure. Although the absorption bandwidth can be expanded according to the method, the method generally leads to a complicated design and an increase in thickness in a case where there is no proper design objective. Therefore, the method is not applicable to confined spaces.

Recent research has also revealed a fundamental constraint on maximum absorption achievable within a given thickness, referred to as a causality constraint. The constraint is derived from a causality principle in wave propagation, that is, a response of a system cannot precede an excitation of the system. The causality constraint provides a theoretical framework for understanding trade-off between an absorption bandwidth and a structural thickness of each of the acoustic metamaterials, guiding the design of an optimal absorber.

Despite these advancements, a systematic method is still needed to design and manufacture the acoustic metamaterial absorber to achieve optimal broadband absorption within a given thickness limit. The method uses the causality constraint to determine a theoretically optimal absorption spectrum and provides a practical means to achieve the spectrum. In addition, the method shall be compatible with an efficient mass production technology, so that the method is widely adopted in the real world.

### SUMMARY OF THE INVENTION

The present disclosure relates to the technical field of sound absorbing materials, and particularly relates to a method for designing a causally optimal broadband acoustic metamaterial absorber and an absorber to satisfy the requirement. The present disclosure introduces a new method for designing and manufacturing a metamaterial absorber to implement causally optimal broadband absorption (COBA), including an integrated optimization technology, a resonator array design, and a mass production method. The present disclosure represents a significant advancement in development of a high-performance and space-saving solution for controlling noise.

The technical solutions of the present disclosure are:

A method for designing a causally optimal broadband acoustic metamaterial absorber is provided, and applied to an absorber with a given noise spectrum S(λ) and an effective thickness limit d, where λ is a wavelength of sound in air. The method includes:
(a) calculating a COBA spectrum A_{COBA}(λ) by solving an optimization problem:

$${\int }_{0}^{∞} S ( λ ) A ( λ ) d λ$$

is maximized based on a constraint condition equation

$$- \frac{1}{4 {π}^{2}} {\int }_{0}^{∞} ln [ 1 - A ( λ ) ] d λ \leq d ,$$

where S(λ) is a signal energy spectrum, and A(λ) is a material independent absorption spectrum; and a solution is given based on A_{COBA}(λ)=1−/(4π²S(λ)) in a case where μ/(4π²S(λ))≤1, otherwise A_{COBA}(λ) equals 0, where μ is determined based on the constraint condition equation, A_{COBA}(λ) is the causally optimal broadband absorption spectrum, and μ is a Lagrange multiplier;
(b) designing an array of resonators, where a mode density [symbol] (ω) of the array of the resonators and a resonance strength r(ω) of the array of the resonators match the COBA spectrum, and a relationship between the mode density and the resonance strength is determined based on the following equation:

$${𝒩}_{d} ( ω ) r ( ω ) = \frac{2 N}{π ϕ ρ c} \frac{{A}_{COBA} ( ω )}{2 + 2 \sqrt{1 - {A}_{COBA} ( ω )} - {A}_{COBA} ( ω )} \qquad ( 1 )$$

where ω is a circular frequency, φ is a surface porosity, ρ is an air density, and c is a sound speed; and
(c) optimizing a structure of the array of the resonators in step (b) and constructing the array of the resonators into the causally optimal broadband acoustic metamaterial absorber.

Further, in the method, the resonator is a quarter-wavelength tube.

Further, in the method, the resonator is a straight structure or a compact structure formed by bending in a case where a length and a cross-sectional area remain unchanged.

Further, in the method, the step (b) further includes obtaining a required mode density and a required resonance strength by adjusting a number, sizes, and a spacing parameter of the resonators.

Further, in the method, optimizing, in the step (c), the structure of the array of the resonators in the step (b) includes making the array of the resonators satisfying a requirement of a mass production method while maintaining a given noise spectrum absorption nature. Further, the mass production method is a molding process method, that is, without affecting acoustic performance, the absorber is produced by demolding from a mold or by assembling components after demolding.

A technical solution of the present disclosure provides a causally optimal broadband acoustic metamaterial absorber obtained by the method.

Further, the acoustic metamaterial absorber includes an array of resonators, and the resonators are quarter-wavelength tubes. A density [symbol] (ωₘ) of a first harmonics of each resonator in a frequency range and an opening area ratio φₘ of each resonator satisfy the following equation:

$${\overline{ℳ}}_{d} ( ω ) = \frac{{A}_{COBA} ( ω ) / 2 ω}{2 + 2 \sqrt{1 - {A}_{COBA} ( ω )} - {A}_{COBA} ( ω )} - {\sum }_{q = 1}^{∞} \frac{{\overline{ℳ}}_{d} [ \frac{ω}{2 q + 1} ]}{{( 2 q + 1 )}^{2}} \qquad ( 2 )$$

where [symbol] (ωₘ)=φₘ [symbol] (ωₘ), and ωₘ is a first circular harmonic frequency of an m-th resonator.

Further, the acoustic metamaterial absorber includes the array of the resonators, and the resonators are the quarter-wavelength tubes, where:
(a) each quarter-wavelength tube has a same opening area; and
(b) a density [symbol] (ω) of a first harmonics of each quarter-wavelength tube in a frequency range satisfies the equation (2), where

$${\overline{ℳ}}_{d} ≡ φ {ℳ}_{d} , φ = {\int }_{{ω}_{1}}^{{ω}_{M}} {\overline{ℳ}}_{d} ( x ) / Mdx$$

is a ratio of an opening area of a single tube to a total area exposed to sound, and M is a number of the quarter-wavelength tubes.

Further, the acoustic metamaterial absorber includes the array of the resonators, and the resonators are the quarter-wavelength tubes, where:
(a) resonant frequencies of all the resonators are evenly distributed with an interval of δ, so that the density [symbol] of the first harmonics of each resonator in the frequency range is a constant and equal to 1/δ;
(b) the opening area ratio φₘ of each resonator satisfies:
φₘ= [symbol] (ωₘ)δ, where [symbol] (ω) is the same as that in the equation (2), and ωₘ is the first circular harmonic frequency of the m-th resonator; and
(c) a first harmonic spacing between the resonators is an integer fraction of a lowest harmonic frequency.

Further, in the acoustic metamaterial absorber, the quarter-wavelength tube is a straight structure or a compact structure formed by bending in a case where a length and a cross-sectional area remain unchanged.

Further, the acoustic metamaterial absorber is manufactured through a molding process.

Further, a method for manufacturing the acoustic metamaterial absorber includes:
(a) designing the absorber by implementing the method for designing the causally optimal broadband acoustic metamaterial absorber; and
(b) manufacturing the designed absorber by using a mass production method.

Further, in the method for manufacturing the acoustic metamaterial absorber, the mass production method is a molding process method, that is, without affecting acoustic performance, the absorber is produced by demolding from a mold or by assembling components after demolding.

Further, in the method for manufacturing the acoustic metamaterial absorber, raw materials suitable for mold production used in mass production include plastic, metal, paper, gypsum, and ceramic.

The present disclosure further provides a technical solution. A system for controlling noise includes one or more causally optimal broadband acoustic metamaterial absorbers or acoustic metamaterial absorbers designed by implementing the method.

Further, an application of the system for controlling the noise is provided. The system for controlling the noise is applied to advanced manufacturing, aerospace, construction, highway and rail transportation, military defense, acoustics, healthcare, energy, environmental protection, entertainment, education, culture and sports, office, home appliances, information technology (IT) or other fields requiring effective noise reduction in confined spaces.

For example, these fields include automobile manufacturing, consumer electronics, audio, sound systems, voice recording devices, home appliance manufacturing, data center facilities, office devices, industrial machinery, shipbuilding, medical devices, energy devices, environmental protection devices, sports equipment, musical instrument manufacturing, toy manufacturing, furniture manufacturing, stage design, music and film production, virtual reality (VR), augmented reality (AR), gaming devices, educational devices, cultural and creative products, and the like.

Compared with related art, the present disclosure has advantages as following.

1. The present disclosure provides a novel design method, in which a mode density and resonance strength distribution of the array of the resonators are adjusted to match a theoretically optimal absorption curve derived from a causality constraint, to obtain a causally optimal broadband absorption absorber that implements a given noise spectrum within a limited thickness. The method overcomes limitations that a traditional porous sound-absorbing material lacks flexibility in customizing an absorption spectrum and has difficulty in absorbing low-frequency sound and a typical acoustic metamaterial exhibits a narrow frequency band absorption nature.

2. The absorber of the present disclosure may be manufactured by using a mass production method, for example, a molding process, and therefore is more suitable for a real application.

3. The present disclosure has potential applications in a plurality of industries, for example, transportation, construction, and manufacturing. Effective noise control in confined spaces is critical in these industries. A framework is provided for designing and manufacturing an acoustic metamaterial absorber with optimal performance. Therefore, the present disclosure represents a significant advancement in the field of noise absorption technologies.

### BRIEF DESCRIPTION OF DRAWINGS

FIG. 1 is a diagram of a comparison between a typical structural scale of a metamaterial absorber and a typical structural scale of a traditional porous material.

FIG. 2 shows a given noise spectrum S(λ) and a COBA spectrum A_{COBA}(λ) optimized under an effective thickness limit d according to an embodiment,
where a is the given noise spectrum S(λ), and b is the COBA spectrum A_{COBA}(λ).

FIG. 3 shows a metamaterial absorber with an array of quarter-wavelength tubes of a same diameter according to an embodiment,
where a is a quarter-wavelength tube with a straight structure, and b is a quarter-wavelength tube in a compact structure formed by folding the tube.

FIG. 4 shows a metamaterial absorber with an array of quarter-wavelength tubes with different diameters and evenly distributed resonant frequencies according to an embodiment.

FIG. 5 shows an absorption spectrum of comparison between performance of a metamaterial absorber customized for noise of a power transformer and performance of a traditional porous material according to an embodiment,
where a is a noise spectrum of a large transformer measured in a one-third octave band, and b is actual absorption spectrum of the metamaterial absorber, actual absorption spectrum of the traditional porous material and the corresponding metamaterial absorber.

### DETAILED DESCRIPTION OF THE INVENTION

Two decades after the initiation of the acoustic metamaterial field, two clear bifurcations are evident: one direction points to continued pursuit of novel phenomenon, mostly through topological structures, and the other points to practical applications on those problems difficult to resolve through conventional means, with an ultimate goal of commercialization. The present disclosure intends to address a latter, in an area of acoustic absorption. Acoustic noise is still a pervasive problem in a 21st century. This is especially the case for low-frequency noise arising from machines, traffic, railroad, airplanes, etc. Such noise may be absorbed by conventional sound-absorbing material, but the required material volume often makes their use impractical. This provides an opportunity for metamaterial absorbers. Can metamaterials do better against the array of low-cost conventional acoustic materials such as foam, rock wool, fiberglass wool, etc.? In recent years, this question has been answered in affirmative. In the description, customizability of the metamaterial is shown, so that the metamaterial enables maximum absorption allowed by causality for any specific noise, with minimum absorber thickness. The resulting absorption performance, in the case of mechanical noise and within confines of limited space, is often far superior to traditional absorbers. On the other hand, a unique structural scale of acoustic metamaterials makes it possible to be mass-produced using a molding process, and the unique structural scale of acoustic metamaterials also allows a diverse selection of materials, for example, metal for high-temperature applications, plastic or paper for lightweight applications, and ceramics for applications requiring high-hardness.

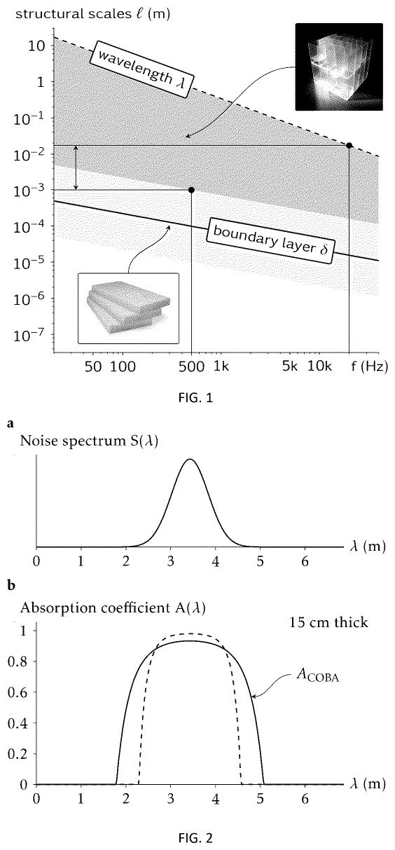

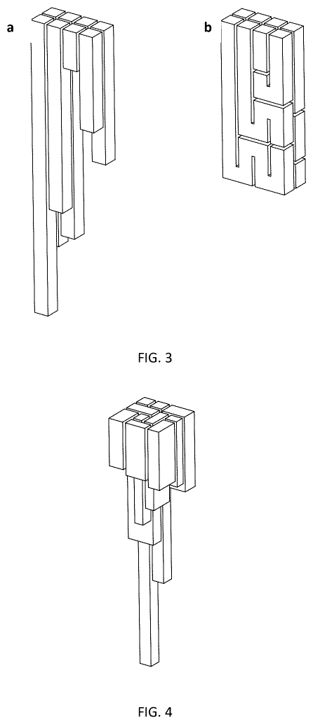

Traditional porous materials, such as foam, rock wool, and fiberglass wool, absorb sound through friction of air molecules at an interface layer between air and a solid skeleton denoted a viscous boundary layer. In the viscous boundary layer, a molecular displacement velocity of air exhibits a monotonic gradient field over a length scale given by δ=\sqrt{2v/(2πf)}, with v=1.5×10⁻⁵ m²/s being a kinetic viscosity of air and f being a sound frequency. Accordingly, a natural way to improve sound absorption efficiency is to increase a solid/air interface area to enhance dissipation efficiency per unit volume. Efficient porous material tends to have porosities close to 1, with an average pore size [symbol] on an order of δ [a light gray region in FIG. 1], so as to maximize the absorption within a given volume. Porous absorbers inherently have a low quality factor, with a broadband absorption spectrum. However, it is to be noted that a dissipation coefficient of the porous absorber shall not be too high. Otherwise, an impedance mismatch, at an interface between air and the absorber, may prevent sound waves from entering the material. Therefore, the impedance mismatch is always a competing consideration for the porous absorber. As impedance is an important parameter for the absorber, its definition and implications are described in detail in the following theory section.

High dissipation coefficient is not only way to increase absorption. An energy absorption density is a product of a material's dissipation coefficient with a local sound energy density. Therefore, a higher energy density can also increase absorption. The metamaterial absorbers essentially take this alternative path. By designing local resonances to increase a local energy density, acoustic metamaterials require only weak material dissipation coefficient, with structural scales [symbol] >>δ. In this way, the absorber can achieve impedance-match with air, with almost 100% absorption. On the other hand, because the metamaterial absorbers' local resonance structures are generally subwavelength in nature with [symbol] <λ=c/f, where c=343 m/s is a speed of sound in air, the structural scales fall in a dark gray shaded region shown in FIG. 1. Metamaterial's low-dissipation and subwavelength nature dictates its absorption to always appear in a form of sparse and narrow frequency peaks with high quality factors. Even though narrow frequency band absorption is meaningful in some special occasions, most of practical applications still require broadband noise absorption capability. To compensate for this inherent defect of the metamaterial absorbers, it is natural to pursue a strategy of integrating multiple units. Each unit resonates at different frequencies, so as to broaden an absorption frequency spectrum. It turns out that there exists an optimal integration scheme for attaining an absorber having broadband, and tunable, absorption spectrum that can surpass performance of traditional acoustic absorbers in defined applications. In other words, a metamaterial absorber may be inversely designed to have a target absorption spectrum, in conjunction with a minimum sample thickness as dictated by the law of natural. In terms of commercialization, this high degree of customizability brings about a paradigm shift in many areas of acoustic applications. The following describes the design scheme in detail starting from a fundamental limitation imposed by causality on wave absorption.

As time can only proceed in one direction, that is, towards the future, the law of causality states that what happens on an absorber at a given instant of time can only depend on what happened before that instant, and cannot depend on what will happen in the future. Mathematically, Fourier transform teaches that time and frequency are conjugate variables. The imposition of the law of causality in the time domain, that is, asymmetry in time, has profound implications for material properties in a frequency domain. In particular, for electromagnetic waves a dielectric function is generally a function of frequency, with real and imaginary components. In the 1920s, two physicists, Hans Kramers and Ralph Kronig, independently derived the famous Kramers-Kronig relation between the real and imaginary parts of a dielectric function. There is also the Bode-Fano bound for network matching. More recent studies also revealed a constraint on the absorption spectrum and a minimum sample thickness r that can be expressed as the following inequality:

$$d \geq - \frac{1}{4 {π}^{2}} {\int }_{0}^{∞} ln [ 1 - A ( λ ) ] d λ = τ \qquad ( 3 )$$

where d is a sample thickness, and A is a ratio of absorbed energy to normally incident energy. An important conclusion from the equation (3) is that perfect absorption cannot exist because A=1 in any finite bandwidth will cause the integral to diverge. This causal inequality also emphasizes importance of the sample thickness as a “resource” for wave absorption. For a given τ, enhanced absorption in one frequency band is usually at the expense of decreased absorption in others; that is, one cannot increase absorption without any cost. Therefore, for a specific signal energy spectrum S(λ), any redundant absorption outside a signal's frequency range is waste, so to speak, and its maximum absorption shall correspond to a material-independent absorption spectrum A(λ) determined only by a thickness of the absorber. This spectrum may be named as causally optimal broadband absorption (COBA) for an incident signal, representing an upper limit of energy absorption allowed by the law of causality. Mathematically, COBA corresponds to maximum in the total absorption defined by

$$E [ A ( λ ) ] ≡ {\int }_{0}^{∞} S ( λ ) A ( λ ) d λ$$

subjecting to constraints of the equation (3) and a given value of d. S(λ) is in the unit of power per unit wavelength.

To find a solution to this optimization problem, a Lagrange multiplier μ is introduced to form a Lagrange functional:

$${E}_{μ} [ A ( λ ) ] ≡ {\int }_{0}^{∞} S A ( λ ) d λ - μ [ \frac{- 1}{4 {π}^{2}} {\int }_{0}^{∞} ln ( 1 - A ( λ ) ) d λ - d ] . \qquad ( 4 a )$$

According to Karush-Kuhn-Tucker conditions, an optimum in E_{μ}[A(λ)] requires the variation δE_{μ}/δA=0, with

$$\{ \begin{matrix}\frac{- 1}{4 {π}^{2}} {\int }_{0}^{∞} ln ( 1 - A ) d λ - d \leq 0 \\ μ > 0 \\ μ [ \frac{- 1}{4 {π}^{2}} {\int }_{0}^{∞} ln ( 1 - A ) d λ - d ] = 0\end{matrix} . \qquad ( 4 b )$$

A theoretical maximum absorption is given by Eₘₐₓ, that is,

$${E}_{max} = {\int }_{0}^{∞} S ( λ ) {A}_{COBA} ( λ ) d λ ,$$

with an ideal A_{COBA} obtained from a condition δE_{μ}/δA=0, that is,

$${A}_{COBA} ( λ ) = \{ \begin{matrix}1 - \frac{μ}{4 {π}^{2}} \frac{1}{S ( λ )} & \frac{μ}{4 {π}^{2}} \frac{1}{S ( λ )} \leq 1 \\ 0 & \frac{μ}{4 {π}^{2}} \frac{1}{S ( λ )} > 1\end{matrix} \qquad ( 5 a )$$

where a second condition ensures A≥0 so as to preserve the conservation of energy. The Lagrange multiplier μ takes the value that satisfies a condition:

$$- \frac{1}{4 {π}^{2}} {\int }_{0}^{∞} ln [ 1 - {A}_{COBA} ( λ ) ] d λ = d . \qquad ( 5 b )$$

This may be done by substituting a solution of the equation (5a) into an integral of the equation (5b). For the given d and the known incident spectrum S(λ), a maximum absorption may be explicitly evaluated.

According to the equation (5), for the given thickness d, each noise signal S(λ) has a unique COBA. The realization of COBA, or the design that targets it, can mean optimal absorber performance at a minimum thickness. As shown in FIG. 2, a Gaussian-type noise signal S(λ)=exp[−(λ−3.43)²] with a center frequency of 100 Hz (is, a center wavelength of 3.43 m) is taken as an example. In a case where the thickness of the absorber is limited to 15 cm, a corresponding A_{COBA} is a solid line in FIG. 2, and total absorption efficiency is

$${\int }_{0}^{∞} SAd λ / {\int }_{0}^{∞} Sd λ = 85.6 \% .$$

Under a same thickness limit, absorption efficiency of any absorption spectrum deviating from the solid line for S(λ) is reduced. For example, a dashed absorption spectrum in FIG. 2 has higher central frequency absorption efficiency within a same thickness of 15 cm, but a total absorption efficiency is reduced to 80.7%.

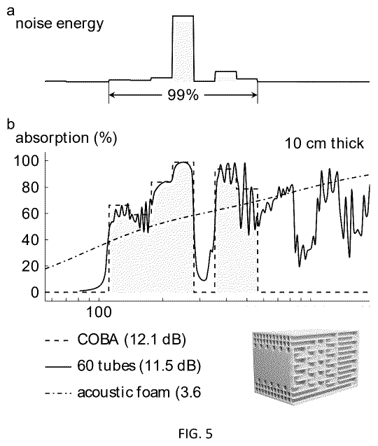

Absorption efficiency (A) of the metamaterial absorber is given by: A=1−|(Z−ρc)/(Z+ρc)|², where ρ=1.2 kg/m³ is an air density, c is the speed of sound in air, Z=p/v̅ is an acoustic surface impedance, with p and v̅ respectively being a pressure modulation and an average normal molecular displacement velocity of air on a surface. For a resonator array facing sound in parallel (as shown in FIG. 3 and FIG. 4), the resonator array's surface impedance Z(ω) may be expressed in terms of a summation of Lorentz functions:

$$Z ( ω ) = \frac{i}{ω} {( \frac{ϕ}{N} {\sum }_{n = 1}^{N} \frac{{f}_{n}}{{ω}_{n}^{2} - {ω}^{2} - i ω β} )}^{- 1} = \frac{i}{ω} {\{ \frac{ϕ}{N} {\sum }_{n = 1}^{N} [ \frac{{f}_{n} ( {ω}_{n}^{2} - {ω}^{2} )}{{{ω}^{2} {β}^{2} + {ω}_{n}^{2} - {ω}^{2} )}^{2}} + \frac{{if}_{n} ωβ}{{ω}^{2} {β}^{2} + {( {ω}_{n}^{2} - {ω}^{2} )}^{2}} ] \}}^{- 1} ,$$

where β is a damping coefficient, rₙ and ωₙ are resonance strength and frequency of a n-th resonator, respectively, N is a total number of resonators, and a surface porosity φ is a ratio of resonators' total opening area to an area exposed to incident sound. For simplicity and generality, a higher-order mode of the resonators is ignored, which may be corrected by a more exact treatment for specific resonator types.

Since a first item of summation of the Lorentz functions changes sign from negative to positive around each resonant frequency, hence summation of all modes tends to cancel out, leading to a negligible net result. In contrast, since a second item of the summation is always positive, contributions of all modes are cumulatively added to each other, resulting in a relatively large value. Therefore, a mode density [symbol] (ω)≡dn/dω may be defined, to convert the summation into an integral, and approximate the impedance in terms of a real integral:

$$Z ( ω ) ≃ {\frac{1}{ω} [ \frac{ϕ}{N} {\int }_{{ω}_{1}}^{{ω}_{N}} \frac{r ( x ) ω β}{{ω}^{2} {β}^{2} + {( {x}^{2} - {ω}^{2} )}^{2}} {𝒩}_{d} ( x ) d x ]}^{- 1} ,$$

where ω₁(ω_{N}) is a frequency of 1st (N-th) resonance. As mentioned above, the metamaterials inherently have high-quality factors, and thus, β is small. This means that the impedance may be further simplified as:

$${lim}_{β \to 0} Z ( ω ) = {\frac{2 N}{ω π ϕ} [ {\int }_{{ω}_{1}}^{{ω}_{N}} \frac{r ( x )}{x} δ ( ω - x ) {𝒩}_{d} ( x ) d x ]}^{- 1} = \frac{2 N}{πϕ} \frac{1}{{𝒩}_{d} ( ω ) r ( ω )} \qquad ( 6 )$$

The equation (6) conveys an important information: The metamaterial absorbers may adjust the impedance by adjusting a product [symbol] (ω)r(ω) of the mode density and a strength function, to implement customized absorption. In an example, for a target optimal absorption spectrum A_{COBA}(ω), as the relevant real Z=ρc(2+2\sqrt{1−A_{COBA}}−A_{COBA})/A_{COBA}, and the suitable [symbol] (ω)r(ω) is given by:

$${𝒩}_{d} ( ω ) r ( ω ) = \frac{2 N}{πϕρ c} \frac{{A}_{COBA} ( ω )}{2 + 2 \sqrt{1 - {A}_{COBA} ( ω )} - {A}_{COBA} ( ω )} \qquad ( 1 )$$

Where the strength function r(ω) generally depends on a specific type of the resonator, and the surface porosity φ is determined by a normalization condition

$${\int }_{{ω}_{1}}^{{ω}_{N}} {𝒩}_{d} ( ω ) d ω = N :$$

$$ϕ = \frac{2}{π ρ c} {\int }_{{ω}_{1}}^{{ω}_{N}} \frac{{A}_{COBA} ( ω ) / r ( ω )}{2 + 2 \sqrt{1 - {A}_{COBA} ( ω )} - {A}_{COBA} ( ω )} d ω .$$

Based on a constraint of the above equation, a suitable array of resonators is obtained, and then the suitable array of resonators constitutes the absorber.

To visualize the consequences of COBA, an example of noise from an electrical transformer is used for study. FIG. 5(a) shows a large transformer's noise measured in a one-third octave bands, in which 99% of energy is concentrated in a frequency range from 110 Hz to 560 Hz. If the thickness of the absorber is limited to τ=10 cm, the equation (5) gives a COBA solution shown in FIG. 5(b) by a dashed line. A relevant

$${E}_{max} / {\int }_{0}^{∞} Sd λ = 93.8 \% ,$$

which is equivalent to 12.1 dB in reflection loss

$$( defined as - 10 {log}_{10} ( 1 - E / {\int }_{0}^{∞} Sd λ ) ) .$$

In contrast, due to a wasted absorption capacity at lower and higher frequencies (a dash-dot line portion in FIG. 5(b)), a traditional acoustic foam with a same 10 cm thickness can only reach efficiency of 56% (3.6 dB in reflection loss).

To test effectiveness of the design and the implementation solution, an acoustic metamaterial absorber with a sample thickness of 10 cm is designed to realize the COBA for the transformer noise. Quarter-wavelength tubes, which are Fabry-Perot (FP) resonators, are used as a fundamental unit. Distribution of resonant frequencies is designed by adjusting lengths of the quarter-wavelength tubes, and a resonance strength is designed by adjusting opening areas of the quarter-wavelength tubes.

In consideration of higher-order resonances, when an array of M FP resonators facing sound in parallel, a surface impedance of the FP resonators is given by:

$$Z ( ω ) = {\frac{i}{ω} [ {\sum }_{m = 1}^{M} {φ}_{m} \frac{tan ( \frac{ω {L}_{m}}{c} )}{ω ρ c} ]}^{- 1} = \frac{i}{ω} {\{ \frac{2}{ρ c} {\sum }_{m = 1}^{M} {\sum }_{q = 0}^{∞} {φ}_{m} \{ \frac{\frac{2 {ω}_{m}}{π}}{{( 2 q + 1 )}^{2} {ω}_{m}^{2} - {ω}^{2}} + i \frac{δ [ ω - ( 2 q + 1 ) {ω}_{m} ]}{2 q + 1} \} \}}^{- 1} \qquad ( 7 )$$

where Lₘ and ωₘ respectively are a length and a first harmonic frequency of an m-th FP tube, q is an order of the harmonics, and φₘ is an areal ratio of an opening area of the m-th FP tube to a total area. The Dirac function in the imaginary part is a result of the Kramers-Kronig relation. If [symbol] (x)dx is introduced to represent a number of FP resonators with a first harmonic frequency x in a frequency range dx, a first summation in the equation (7) may be rewritten as an integral, and, by taking the similar approximation as before to ignore the imaginary part of Z(ω).

$$Z ( ω ) ≃ \frac{1}{ω} {\{ \frac{2}{ρ c} {\sum }_{q = 0}^{∞} \frac{1}{2 q + 1} {\int }_{{ω}_{1}}^{{ω}_{M}} δ [ ω - ( 2 q + 1 ) x ] φ ( x ) {ℳ}_{d} ( x ) d x \}}^{- 1} = \frac{1}{ω} {\{ \frac{2}{ρ c} {\sum }_{q = 0}^{∞} \frac{1}{{( 2 q + 1 )}^{2}} {\overline{ℳ}}_{d} ( \frac{ω}{2 q + 1} ) \}}^{- 1} \qquad ( 7 )$$

where [symbol] ≡φM_{d}. To target the optimal absorption spectrum A_{COBA}(ω), [symbol] can be solved through the iterations following equation:

$${\overline{ℳ}}_{d} ( ω ) = \frac{{A}_{COBA} ( ω ) / 2 ω}{2 + 2 \sqrt{1 - {A}_{COBA} ( ω )} - {A}_{C O B A} ( ω )} - {\sum }_{q = 1}^{∞} \frac{{\overline{ℳ}}_{d} [ \frac{ω}{2 q + 1} ]}{{( 2 q + 1 )}^{2}} \qquad ( 2 )$$

A simple example is that every FP tube shares a same opening area. φ is a constant, and the mode density [symbol] (ω) of the FP resonators is designed. Because of the normalization condition,

$${\sum }_{m = 1}^{M} {ℳ}_{d} = M ,$$

which requires that

$${\int }_{{ω}_{1}}^{{ω}_{M}} {ℳ}_{d} ( x ) d x = M ,$$

and an area ratio φ may be determined by:

$$φ = {\int }_{{ω}_{1}}^{{ω}_{M}} \frac{{\overline{ℳ}}_{d} ( x )}{M} d x .$$

To further determine the first harmonics, thus the lengths, of the FP tubes, dm̅≡dm/M and

$$\overline{m} ∈ [ \frac{1}{M} , 1 ]$$

are introduced. According to a definition of [symbol] , φMdm̅= [symbol] (ω)dω, hence:

$$\overline{m} ( x ) = \frac{1}{φ M} {\int }_{{ω}_{1}}^{x} {\overline{ℳ}}_{d} ( ω ) d ω + \frac{1}{M}$$

By using a plotting of m̅(x), discrete resonators in an actual design may be easily determined by locating the first harmonics of the FP tubes on a horizontal axis with the associated values of m̅ (equally spaced on a vertical axis).

As shown in FIG. 3, the designed absorber includes an array of resonators, and the resonators are quarter-wavelength tubes, where:
(a) each quarter-wavelength tube has a same opening area; and
(b) a density [symbol] (ω) of a first harmonics of each quarter-wavelength tube in a frequency range satisfies the equation (2) due to distribution of lengths of the tubes, where

$${\overline{ℳ}}_{d} ≡ {φℳ}_{d} , φ = {\int }_{{ω}_{1}}^{{ω}_{M}} {\overline{ℳ}}_{d} ( x ) / Mdx$$

is an areal ratio of an opening area of a single tube to a total area exposed to sound, and M is a number of quarter-wavelength tubes.

The quarter-wavelength tube may be straight (FIG. 3(a)) or a compact structure (FIG. 3(b)) formed by bending in a case where a length and a cross-sectional area remain unchanged.

Another simple example is that all resonant frequencies are evenly distributed with an interval of δ. So that, [symbol] =1/δ is a constant, and a cross-sectional area of each FP tube shall be designed to satisfy φₘ= [symbol] (ωₘ)δ. It is to be noted that, in consideration of higher-order harmonics of FP tubes, to make sure all the resonant frequencies can be evenly distributed, the spacing of the FP tubes' first harmonics shall be an integer fraction of a lowest harmonics.

As shown in FIG. 4, the absorber also includes an array of resonators, each of which is a quarter-wavelength tube, where:
(a) resonant frequencies of all the resonators are evenly distributed with an interval of δ, so that the density [symbol] of the first harmonics of each resonator in the frequency range is a constant and equal to 1/δ;
(b) a surface area ratio φₘ of each resonator is designed to satisfy φₘ= [symbol] (ωₘ)δ, where [symbol] (ω) satisfies the equation (2), and ωₘ is a first circular harmonic frequency of the m-th resonator; and
(c) a first harmonic spacing between the resonators is an integer fraction of a lowest harmonic frequency.

The transformer's noise is still used as an example. A first strategy that the array of the FP tubes has a same opening area is used. As shown in FIG. 5(b), 60 folded FP tubes are produced within a space of 9×9×10 cm³ through a molding process, to form a compact and maze-like structure as a functional unit of the metamaterial absorber. In the laboratory impedance tube measurement, an absorption spectrum delineated by a solid line in FIG. 5(b) is shown. It can be seen that an experimental measurement result, while highly undulating in character, follows closely the COBA solution in the frequency range from 110 Hz to 560 Hz, where the noise energy is concentrated. A total reflection loss of the tubes is 11.5 dB, which is only 0.6 dB lower than that of COBA and much higher than that of the traditional acoustic foam. A discrepancy with COBA arises mainly come from an unavoidable higher-order modes of the FP tubes which absorb sound higher than 560 Hz. However, higher frequency absorption contributes very little to the causal constraint integral of the equation (3). In an actual application, a large number of such metamaterial absorbers may be combined into a sound absorbing panel, a soundproof wall, or a soundproof cover, thereby providing systematic noise reduction for a target machine. Further, these metamaterial absorbers may be respectively customized for different noise spectra in different regions of the machine, so that these different metamaterial absorbers are used at corresponding positions and combined to form a more efficient overall solution.

The example indicates that absorption performance of customized COBA is generally far better than that of a traditional porous materials, especially when the noise has a large low-frequency component. The advantage is a cornerstone for commercialization of the acoustic metamaterials. However, intricate structures of the metamaterial absorbers pose a challenge in mass production. While flexibility of 3D printing makes it ideal for prototyping and testing, an existing 3D printing technology still faces challenges of production efficiency and yield rate in mass production. Fortunately, as shown in FIG. 1, because the sound wavelengths are not very small for audible acoustics [20 Hz to 20,000 Hz], acoustic metamaterial's structural scales are usually on the order of millimeters to centimeters, which falls within a range of a traditional mold production process, which is more efficient, reliable, and more suitable for mass production. A photo in an illustration on an upper right side of FIG. 1 shows a COBA metamaterial absorber produced through mold injection. A layered structure in an illustration in FIG. 5(b) is an optimized structure for the mold production method. In this way, without affecting acoustic performance, the metamaterial absorber may be produced by demolding or by assembling components after demolding. In addition, the mass production method for the mold may be implemented through plastic injection and may also use other raw materials suitable for mold production, including metal, paper, plaster, and ceramic.

The present disclosure further provides another example of a system for controlling noise including one or more acoustic metamaterial absorbers designed by implementing the method, or one or more causally optimal broadband acoustic metamaterial absorbers.

The system for controlling the noise may be applied to advanced manufacturing, aerospace, construction, highway and rail transportation, military defense, acoustics, healthcare, energy, environmental protection, entertainment, education, culture and sports, office, home appliances, information technology (IT), or other fields requiring effective noise reduction in confined spaces.

Specifically, these fields include automobile manufacturing, consumer electronics, audio, sound systems, voice recording devices, home appliance manufacturing, data center facilities, office devices, industrial machinery, shipbuilding, medical devices, energy devices, environmental protection devices, sports equipment, musical instrument manufacturing, toy manufacturing, furniture manufacturing, stage design, music and film production, virtual reality (VR), augmented reality (AR), gaming devices, educational devices, cultural and creative products, and the like.

1. A method for designing a causally optimal broadband acoustic metamaterial absorber, applied to an absorber with a given noise spectrum S(λ) and an effective thickness limit d, wherein λ is a wavelength of sound in air, and the method comprises:
(a) calculating a causally optimal broadband absorption (COBA) spectrum A_{COBA}(λ) by solving an optimization problem:
$${\int }_{0}^{∞} S ( λ ) A ( λ ) d λ$$
is maximized based on a constraint condition equation
$$- \frac{1}{4 {π}^{2}} {\int }_{0}^{∞} ln [ 1 - A ( λ ) ] d λ \leq d ,$$
wherein S(λ) is a signal energy spectrum, and A(λ) is a material independent absorption spectrum; and a solution is given based on A_{COBA}(λ)=1−μ/(4π²S(λ)) in a case where μ/(4π²S(λ))≤1, otherwise A_{COBA}(λ) equals 0, wherein μ is determined based on the constraint condition equation, A_{COBA}(λ) is the causally optimal broadband absorption spectrum, and μ is a Lagrange multiplier;
(b) designing an array of resonators, wherein a mode density [symbol] (ω) of the array of the resonators and a resonance strength r(ω) of the array of the resonators match the COBA spectrum, and a relationship between the mode density and the resonance strength is determined based on the following equation:
$${𝒩}_{d} ( ω ) r ( ω ) = \frac{2 N}{πφρ c} \frac{{A}_{C O B A} ( ω )}{2 + 2 \sqrt{1 - {A}_{C O B A} ( ω )} - {A}_{C O B A} ( ω )} \qquad ( 1 )$$
wherein ω is a circular frequency, φ is a surface porosity, ρ is an air density, and c is a sound speed; and
(c) optimizing a structure of the array of the resonators in step (b) and constructing the array of the resonators into the causally optimal broadband acoustic metamaterial absorber.

2. The method according to claim 1, wherein the resonator is a quarter-wavelength tube.

3. The method according to claim 1, wherein the resonator is a straight structure.

4. The method according to claim 1, wherein the step (b) further comprises obtaining a required mode density and a required resonance strength by adjusting a number, sizes, and a spacing parameter of the resonators.

5. The method according to claim 1, wherein optimizing, in the step (c), the structure of the array of the resonators in the step (b) comprises making the array of the resonators satisfying a requirement of a mass production method while maintaining a given noise spectrum absorption nature.

6. A causally optimal broadband acoustic metamaterial absorber, wherein the causally optimal broadband acoustic metamaterial absorber is obtained by implementing the method according to claim 1.

7. The acoustic metamaterial absorber according to claim 6, wherein the absorber comprises an array of resonators, and the resonators are quarter-wavelength tubes, wherein a density [symbol] (ωₘ) of a first harmonics of each resonator in a frequency range and an opening area ratio φₘ of each resonator satisfy the following equation
$${\overline{ℳ}}_{d} ( ω ) = \frac{{A}_{C O B A} ( ω ) / 2 ω}{2 + 2 \sqrt{1 - {A}_{C O B A} ( ω )} - {A}_{C O B A} ( ω )} - {\sum }_{q = 1}^{∞} \frac{{\overline{ℳ}}_{d} [ \frac{ω}{2 q + 1} ]}{{( 2 q + 1 )}^{2}} \qquad ( 2 )$$
wherein [symbol] (ωₘ)=φₘ [symbol] (ωₘ), and ωₘ is a first circular harmonic frequency of an m-th resonator.

8. The acoustic metamaterial absorber according to claim 7, wherein the absorber comprises the array of the resonators, and the resonators are the quarter-wavelength tubes, wherein
(a) each quarter-wavelength tube has a same opening area; and
(b) a density [symbol] (ω) of a first harmonics of each quarter-wavelength tube in a frequency range satisfies the equation (2), wherein
$${\overline{ℳ}}_{d} ≡ {φℳ}_{d} , φ = {\int }_{{ω}_{1}}^{{ω}_{M}} {\overline{ℳ}}_{d} ( x ) / Mdx$$
is a ratio of an opening area of a single tube to a total area exposed to sound, and M is a number of the quarter-wavelength tubes.

9. The acoustic metamaterial absorber according to claim 7, wherein the absorber comprises the array of the resonators, and the resonators are the quarter-wavelength tubes, wherein
(a) resonant frequencies of all the resonators are evenly distributed with an interval of δ, so that the density [symbol] of the first harmonics of each resonator in the frequency range is a constant and equal to 1/δ;
(b) the opening area ratio φₘ of each resonator satisfies:
φₘ= [symbol] (ωₘ)δ, wherein [symbol] (ω) is the same as that in the equation (2), and ωₘ is the first circular harmonic frequency of the m-th resonator; and
(c) a first harmonic spacing between the resonators is an integer fraction of a lowest harmonic frequency.

10. The acoustic metamaterial absorber according to claim 7, wherein the quarter-wavelength tube is a straight structure.

11. The acoustic metamaterial absorber according to claim 6, wherein the absorber is manufactured through a molding process.

12. A method for manufacturing the acoustic metamaterial absorber according to claim 6, comprising:
manufacturing the designed absorber by using a mass production method.

13. The method for manufacturing the acoustic metamaterial absorber according to claim 12, wherein the mass production method is a molding process method.

14. The method for manufacturing the acoustic metamaterial absorber according to claim 12, wherein raw materials suitable for mold production used in mass production comprise plastic, metal, paper, gypsum, and ceramic.

15. A system for controlling noise, comprising one or more acoustic metamaterial absorbers designed by implementing the method according to claim 1.

16. An application of the system for controlling the noise according to claim 15, wherein the system for controlling the noise is applied to advanced manufacturing, aerospace, construction, highway and rail transportation, military defense, acoustics, healthcare, energy, environmental protection, entertainment, education, culture, sports, office, home appliances, information technology (IT) or other fields requiring effective noise reduction in confined spaces.

17. The method according to claim 1, wherein the resonator is a compact structure formed by bending in a case where a length and a cross-sectional area remain unchanged.

18. The acoustic metamaterial absorber according to claim 7, wherein the quarter-wavelength tube is a compact structure formed by bending in a case where a length and a cross-sectional area remain unchanged.

19. A system for controlling noise, comprising one or more causally optimal broadband acoustic metamaterial absorbers according to claim 6.

20. An application of the system for controlling the noise according to claim 19, wherein the system for controlling the noise is applied to advanced manufacturing, aerospace, construction, highway and rail transportation, military defense, acoustics, healthcare, energy, environmental protection, entertainment, education, culture, sports, office, home appliances, information technology (IT) or other fields requiring effective noise reduction in confined spaces.
